(19) United States
(12) Plant Patent Application Publication
(10) Pub. No.: US 2005/0188445 P1
(43) Pub. Date: Aug. 25, 2005

(54) Chrysanthemum plant named 'Sizzleness'

(50) CHRYSANTHEMUM MORIFOLIUM; SIZZLENESS

(76) Inventor: Mark Roland Boeder, The Hague (NL)

Correspondence Address: PARKHURST & WENDEL, SUITE 210, 1421 PRINCE STREET, ALEXANDRIA, VA, 22314, US

(21) Appl. No.: 10/782,856

(22) Filed: Feb. 23, 2004

(51) Int. Cl. A01H 5/00

(52) U.S. Cl. PLT/287

(57) ABSTRACT

A Chrysanthemum plant named ‘Sizzleness’ characterized by its multicolored medium sized blooms with pink/cream ray-florets and green disc florets, with a response time of 52 days.

### BACKGROUND OF THE INVENTION

‘Sizzleness’ is a product of a breeding-program which had the objective of creating new chrysanthemum cultivars with a decorative type flower, a 7.5 week response and a medium plant height (1 m). The new plant of the present invention comprises a new and distinct cultivar of Chrysanthemum plant. ‘Sizzleness’ is a seedling from a cross in a breeding program maintained under the control of inventor. The female parent is #96.1260 and the male parent #97.0324, both unpatented plants form the proprietary seedling collection of the inventor. The new and distinct cultivar was discovered and selected as a flowering plant within the progeny of the stated cross by Mark Roland Boeder in a controlled environment (greenhouse) in Rijsenhout Holland in 2001.The first act of asexual reproduction of ‘Sizzleness’ was accomplished when vegetative cuttings were taken from the initial selection in 2001 in a controlled environment in Rijsenhout Holland.

### SUMMARY OF THE INVENTION

The present invention is a new and distinct variety of chrysanthemum bearing medium sized multicolored blooms with pink/cream ray-florets and green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

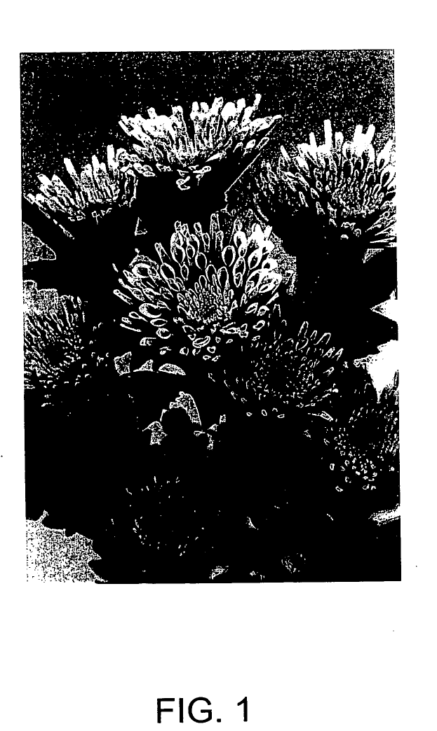

FIG. 2 shows the various stages of bloom of the new cultivar.

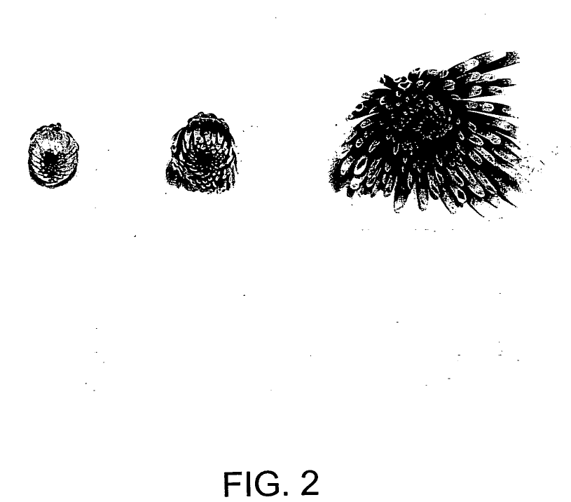

FIG. 3 shows the foliage of the new cultivar.

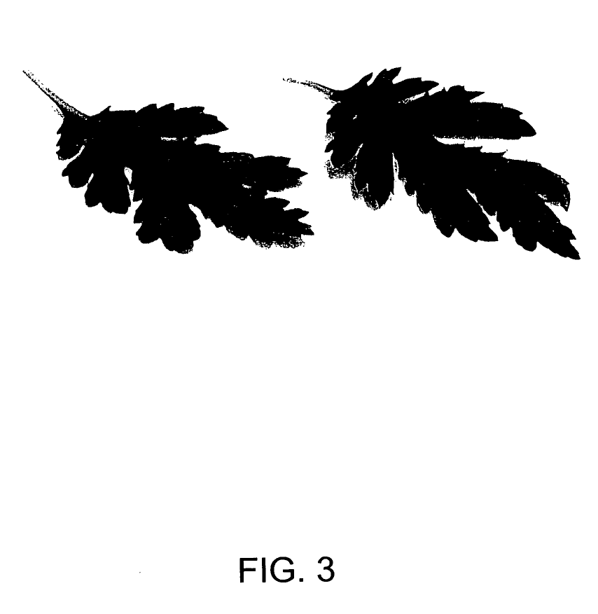

### DESCRIPTION OF THE INVENTION

This new variety of chrysanthemum is of the botanical classification Chrysanthemum morifolium. The observations and measurements were gathered from plants grown in April/May in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 52 days. After the long day period to flowering growth retardants were applied 2 to 3 times in an average dose of 2.5 gram/liter water. The plants were observed (directly) during the flowering of this crop. No tests were done on disease or insect resistance or susceptibility. No tests were done on cold or drought tolerance. This new variety produces medium sized blooms with pink/cream ray-florets and green disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘Sizzleness’ can be planted without assimilation lightning (high pressure sodium lamps) between week 26 and week 16 of the next year under greenhouse conditions in Holland. With assimilation light (minimum level 2500 lux) it can be planted year round under greenhouse conditions in Holland.

From the cultivars known to inventor the most similar existing cultivar in comparison to ‘Sizzleness’ is ‘Spring’ (U.S. Plant Pat. No. 12,934). When ‘Spring’ and ‘Sizzleness’ are being compared the following differences and similarities are noticed.

The inflorescence of both varieties consists of a small center of disc florets, surrounded by several rows of quilled ray florets. The differences of ‘Spring’ and ‘Sizzleness’ are (I) Number ray florets. The number is in ‘Spring’ larger than in ‘Sizzleness’. (2) Color ray florets. The color is in ‘Spring’ pink, while this is dark pink with a cream edge in ‘Sizzleness’.

The following is a description of the plant and characteristics that distinguish ‘Sizzlenesss’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

TABLE 1
Botanical Description of cultivar ‘Sizzleness’

| | |
|---|---|
| Bud | |
| Size | Medium, cross-section 0.6 cm height 1 cm |
| Outside color | Yellow-green 150D/Purple-violet 80D |
| Involucral bracts | 2 rows, length 7 mm, width 3 mm |
| Involucral bracts among | Not present |
| disc-florets | |
| Involucral bracts color | Green 138B |
| Bloom | |
| Type | Decorative |
| Height | 3 cm |
| Size | Medium |
| Fully expanded | 6 cm |
| Number of blooms per stem | Average of 18-20 |
| Performance on the plant | 5 weeks |
| Seeds (if crossed) | Produced in small quantities, oval shaped, |
| | grey-brown 199A, 2 mm in length. |
| Fragrance | Typical chrysanthemum, slight |
| Peduncle length | Near the top 14 cm, near the middle 13 cm, |
| | near the bottom 12 cm |
| Peduncle color | Yellow-green 148A |
| Color | |
| Center of the flower | Immature Yellow-green 149A |
| | Mature Yellow-green 154C |
| Color of the ray-florets | Upper surface Purple-violet 80C/Yellow 4D |
| | Lower surface Purple-violet 80D/Yellow 4D |
| Tonality from Distance | A spray mum with pink/cream flowers and |
| | green disc |
| Color of upper side of ray | Purple-violet 80C with Yellow 4D at edges |
| florets at aging of the plant | |
| Ray florets | |
| Texture | Upper and under side smooth |
| Number | 100 |
| Shape | Elongated quilled |
| Longitudinal axis of | Straight |
| majority | |
| Length of corolla tube | Long; 2.5 cm |
| Ray-floret margin | Entire |
| Ray-floret length | 3 cm |
| Ray-floret width | 0.3 cm |
| Ratio length/width | High |
| Shape of tip | Rounded |
| Disc florets | |
| Disc diameter | 0.8 cm |
| Distribution of disc florets | Few, only visible in mature stage of |
| | flowering |
| Shape | Tubular |
| Color | Yellow-green 154C |
| Receptacle shape | Domed raised |
| Reproductive Organs | |
| Stamen (present in | Thin |
| disc florets only) | |
| Stamen color | Yellow-green 144 B |
| Pollen | Produced in small quantity |
| Pollen color | Yellow 7A |
| Styles (present in both | Thick |
| ray and disc florets) | |
| Style color | Yellow-green 150C |
| Style Length | 4 mm |
| Stigma color | Yellow-green 145 D |
| Stigma Width | 1 mm |
| Ovaries | Enclosed in calyx |
| Plant | |
| Form | A spray mum meant for erect culture |
| Growth habit | Upright |
| Growth rate | Medium vigor |
| Height | 90 cm |
| Width | 28 cm |
| Internode lenght | 2-3.5 cm |
| Spray formation | Cylindrical |
| Stem Color | Yellow-green 148A |
| Stem Strength | Strong |
| Stem Brittleness | Not brittle |
| Stem Anthocyanin | Absent |
| coloration | |
| Flowering Response | 52 days |
| (photo-periodic controlled | |
| crop, not natural season) | |
| Foliage | |
| Color immature stage | Upper side Green 137B |
| | Under side Green 138C |
| Color mature stage | Upper side Green 137A |
| | Under side Green 138A |
| Color midvein mature leaf | Upper side Yellow-green 145C |
| | Under side Yellow-green 148C |
| Size | Medium; length 15 cm, width 7.5 cm |
| Quantity (number per | 30 |
| single stem) | |
| Shape | Elliptic |
| Texture upper side | Fleshy and glabrous |
| Texture under side | Pubescent |
| Venation arrangement | Palmate |
| Shape of the margin | Dentate |
| Shape of Base of Sinus | Diverging |
| Between Lateral Lobes | |
| Margin of Sinus Between | Rounded |
| Lateral Lobes | |
| Shape of Base | Obtuse |
| Apex | Mucronulate |
| Petiole Length | 2 cm |
| Petiole Color | Yellow-green 145C |

TABLE 2
Differences with the comparison variety

| | ‘Sizzleness’ | ‘Spring’ |
|---|---|---|
| Number of ray florets | 100 | 224 |
| Color of ray florets | Purple-violet 80C with | Purple 75B to 75C |
| | Yellow 4D at edges | |

1. A new and distinct variety of chrysanthemum plant as described and illustrated.
